(19) United States
(12) Patent Application Publication
(10) Pub. No.: US 2008/0183034 A1
(43) Pub. Date: Jul. 31, 2008

(54) Endoscope Stand

(75) Inventors: Hezi HENKIN, Even Yehuda (IL); Adi STRAUSS, Haifa (IL); Harry PIASKOWSKY, Raanana (IL); Israel HERSKOWITZ, Raanana (IL)

Correspondence Address: Sean Liam Kelleher;Kelleher & Lilling PLLC, P.O. BOX 560, GOLDEN BRIDGE, NY, 10526, US

(73) Assignee: STRYKER GI LTD., Caesarea (IL)

(21) Appl. No.: 12/017,962

(22) Filed: Jan. 22, 2008

Related U.S. Application Data
(60) Provisional application No. 60/887,511, filed on Jan. 31, 2007.

(51) Int. Cl. A61B 19/00

(52) U.S. Cl. 600/102

(57) ABSTRACT

A stand for supporting at least an operating handle and an insertion tube of an endoscopic apparatus is disclosed. The stand includes a hanger member and a post member. Said hanger member has a first shoulder portion adapted for supporting an umbilical cord of the endoscopic apparatus, an intermediate portion adapted for supporting the operating handle, a carrier portion adapted for supporting the insertion tube and a second shoulder portion adapted for retaining a distal end of the insertion tube in an upright position.

### FIELD OF THE INVENTION

The present invention refers to endoscopy, and in particular to colonoscopy and gastroscopy or any other endoscopic procedure in the course of which an endoscope is placed on a stand before performing the endoscopic procedure or after completing the endoscopic procedure. More specifically, the invention refers to a stand for retaining endoscopes fitted with operating handles for navigation and with disposable components, like a sleeve for protecting the insertion tube, a disposable cassette for storing the sleeve before performing the endoscopic procedure and a multichannel for supplying fluid medium to the operating handle.

### BACKGROUND OF THE INVENTION

Stands for holding endoscopes and colonoscopes are known and described for example in Takase (U.S. Pat. No. 6,716,159), Takahashi (U.S. Pat. No. 5,337,731), Elsie (US Patent Publication 2006/0235268), Frassica (U.S. Pat. No. 5,520,607), and Adams (U.S. Pat. No. 4,620,527).

Endoscopes provided with various disposable items like disposable protective sleeves and disposable cassettes, as well as peculiarities of preparation of such endoscopes for the endoscopic procedure, are described in Bar-Or (WO 2005/110204, International patent application PCT/IL2005/000425; and WO 2005/110185, International patent application PCT/IL2005/000426). An example of a disposable cassette for use with such endoscopes is described in Aizenfeld (US Patent Publication 2007/0249902).

There are known conventional “vertical” stands. They are suitable for supporting the operating handle while the insertion tube of an endoscope is hanging with its distal end facing down. Those stands are not capable of providing support for the insertion tube or for the cassette. Those stands are not designed for retaining the distal end of the insertion tube in the erected position or for supporting the cassette when it is in the upright position.

There are also known “horizontal” stands, which are designed to support the operating handle and the insertion tube while both are being directed horizontally.

The disadvantages of the known in the art supporting stands are as follows.

Conventional “vertical” stands do not prevent falling of the operating handle.

Conventional “vertical” stands are not useable with endoscopes employing disposable sleeves stored in a cassette.

Conventional “horizontal” stands require space and are not suitable for relatively small operating rooms and procedural rooms.

### SUMMARY OF THE INVENTION

The object of the invention is to provide an endoscope with a supporting stand, which allows safe and reliable hanging of the operating handle, such that the handle is prevented from falling from the stand.

A further object of the invention is to provide the endoscope with a stand, which allows supporting the distal end of the bendable portion of the insertion tube in an erected position and retaining the disposable cassette in a substantially vertical, upright position, such that any inadvertent spilling out of the lubricant from the cassette is prevented.

To achieve the above objects the stand of the invention is provided with a hanger member and with a post member securable at any side of the system control unit. The hanger member is fitted with a first shoulder portion for supporting the umbilical cord and for supporting the multichannel, with an intermediate portion for supporting the operating handle, with a carrier portion and with a second shoulder portion for securing the bendable section of the insertion tube, for retaining the distal end of the insertion tube in an erect position and for supporting the disposable cassette in the upright position. The intermediate portion is located between the first shoulder portion and the carrier portion. The intermediate portion is rigidly connected to the first shoulder portion and to the carrier portion. In an alternate embodiment of the invention the second shoulder portion is detachably connected to the carrier portion. In a further embodiment of the invention the second shoulder portion can be pivoted with respect to the carrier portion.

The first shoulder portion is configured as a gutter, which might have an elongate window made in the bottom of the gutter.

The intermediate portion is provided with a recess for receiving the handle. The recess is opened from below to allow passing therethrough of the insertion tube. The recess is provided with a support region for receiving the control buttons of the handle and with vertical walls, which provide lateral support for the control buttons portion and thus prevent the handle from falling due to inadvertent tilting. The recess, the support region and the vertical walls are configured and dimensioned to allow receiving of any operating handle irrespective of its manufacturing size. The intermediate portion is securable on the post member by a threaded detent knob provided at a rear side of the hanger member.

The second shoulder portion is designed as a gutter, which is shaped to support the insertion tube. The second shoulder portion is designed not to disturb the dismantling of the cassette when it is required in the course of the colonoscopic procedure, carried out with the colonsocopic apparatus provided with a disposable sleeve and cassette for storing the sleeve. The gutter has an arched shape and its radius of curvature as well as its cross-sectional configuration is selected to provide sufficient friction between the gutter and the insertion tube and thus to allow reliable supporting of the insertion tube on the second shoulder portion without sliding. The second shoulder is provided with a clamp piece, which is made of resilient material and is designed to be detachably affixed thereto. The clamp piece is provided with a rounded cut-out, whose dimension and configuration are selected to allow forcible insertion of the insertion tube and its clamping due to the resiliency of the clamp piece material. By virtue of the clamp piece the insertion tube is secured on the second shoulder portion. The second shoulder portion is detachably connected to the carrier portion and upon connection is carried thereby. In accordance with an alternate embodiment the second shoulder portion can pivot with respect to the carrier portion around a vertical axis, which is directed perpendicularly to the carrier portion. The second shoulder portion is securable on the carrier portion by a dedicated threaded detent knob.

The second shoulder portion is provided with a shelf arrangement for placing the cassette thereon and supporting the cassette when it is in an upright position.

### BRIEF DESCRIPTION OF THE DRAWINGS

FIG. 1 and FIG. 2 show a colonoscopic system employing a stand

FIGS. 3, 4 and 5 show isometric views of a hanger member employed in the present invention.

FIG. 6 shows an intermediate portion of the hanger member.

FIG. 7 shows the operating handle being received in the intermediate portion.

FIG. 8 shows an endoscope resting on the stand of the present invention.

FIGS. 9 and 10 show a second embodiment of the hanger member.

FIGS. 11-15 show how a second shoulder portion of the hanger member is being pivotally displaced from one position to another position.

### DETAILED DESCRIPTION OF THE INVENTION

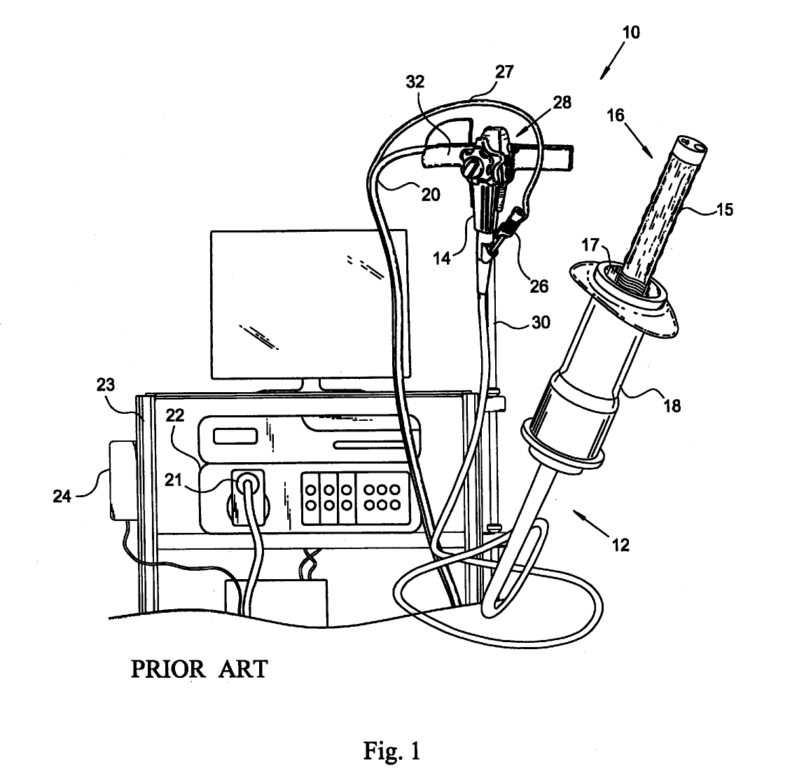

FIG. 1 is a general view of a colonsocopic apparatus provided with disposable components when its operating handle is hung on a prior art stand.

A colonoscopic apparatus 10 is shown with its following main components. The apparatus comprises an endoscope having an insertion tube with its proximal section 12 connected to an operation handle 14 and with its distal section 16 inserted in and protruding from a disposable dispenser or cassette 18. An example of such an apparatus and explanation of its construction and functioning can be found in Eizenfeld (WO 2004/016299, International patent application PCT/IL2003/000661) and Bar-Or (WO 2005/110204, International patent application PCT/IL2005/000425).

A disposable inflatable protection sleeve covers the distal region of the endoscope. That part of the sleeve, which is seen in FIG. 1, comprises a frontal noninflatable portion 15 and a rear folded portion 17. The frontal noninflatable portion 15 covers the distal section 16 of the endoscope and its head. During the procedure when the endoscope advances within the colon, the frontal portion 15 does not inflate, whereas the folded rear portion 17, which before the procedure is stored in the cassette 18, unfolds when air, or other fluid medium, is pumped into and inflates the sleeve. The endoscope is propelled within the body passage when the sleeve is being inflated. To ensure easy unfolding of the sleeve and its reliable feeding out from the cassette a liquid lubricant is supplied to the cassette before initiating the procedure. This is described for example in Bar-Or (WO 2005/110185, International patent application PCT/IL2005/000426).

It is seen also in FIG. 1, that the operating handle is connected by an umbilical cord 20 to a multifunctional connector 21, which is plugged into a system control unit (SCU) 22. The SCU is located on a cart 23. The SCU is provided with appropriate sources of fluid media, like compressed air or other gas for inflating the sleeve and for insufflation the body passage. Furthermore, a source of water, e.g. an irrigation flask 24, is provided at the SCU. This flask is filled with water or other liquid, to be supplied via the insertion tube into the colon for irrigation or for other purposes. A vacuum source is also provided, being either a part of the SCU or a dedicated separate source, e.g. hospital equipment. It is not seen in FIG. 1, but should be appreciated, that along the insertion tube extends a multilumen tubing with appropriate passages for supplying water, as required for irrigation of the colon, air as required for insufflation and vacuum as required for suction.

The multilumen tubing also allows introduction of surgical instruments into the colon as might be required during a colonoscopic procedure. The multilumen tubing extends through the entire length of the insertion tube, passes the handle and is connected to a dedicated connector 26, which is detachably connectable to a lateral port provided on the handle, so as to connect the proximal end of the multilumen tubing with ducts of a multichannel 27 extending along the umbilical cord. The multichannel supplies fluid medium from the SCU to the corresponding ducts of the multilumen tubing

The colonoscopic apparatus is provided with a stand, which comprises a hanger member 28 and a post member 30. The hanger member is secured at a certain height on the post member so as to enable free hanging of the endoscope on the stand at a convenient height. The hanger member is provided with a shoulder portion 32 for supporting the umbilical cord and the multichannel. The prior art stand is suitable merely for hanging thereon of the operating handle. It is not designed for supporting the insertion tube with its distal section being in the erected position as would be required for preventing the cassette from detachment from the insertion tube and for preventing the lubricant from spilling out from the cassette. For this purpose an operator should manually hold the insertion tube and the cassette, such that the cassette would be in the upright position.

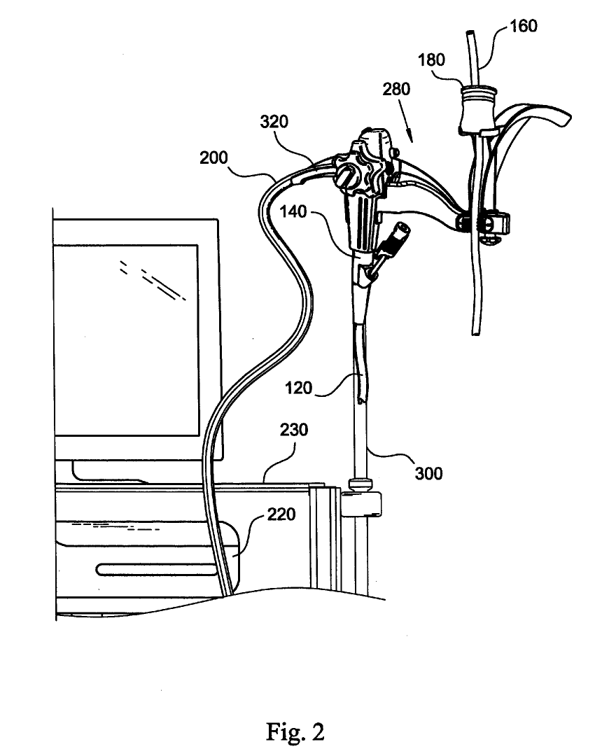

FIG. 2 shows a perspective view of the colonsocopic apparatus of the invention, which comprises a colonoscope similar to that shown in FIG. 1 and provided with the new stand of the herein invention. The new stand is suitable for hanging the operating handle, for retaining the distal section of the insertion tube in the erected position and for supporting the cassette when it is in the up-right position.

For convenience those elements of the colonoscopic apparatus shown in FIG. 2 which are similar to the elements shown in FIG. 1 are designated by the same reference numerals however with an added zero. So the stand of the invention comprises a hanger member 280, securable on a post member 300. The post member is secured on a cart 230 at a side of an SCU 220. The hanger member is provided with a shoulder portion 320 for carrying an umbilical cord 200. As shown in FIG. 2 by virtue of the stand of the present invention it is possible to hang an operating handle 140 of the colonoscope such that a proximal section 120 of the insertion tube will be directed down, while a distal section 160 of the insertion tube is in an erected position such that its distal section will be facing up. It is also shown that by virtue of the stand of the invention a cassette 180 is supported from below and is retained in the up-right position such that its detachment from the insertion tube as well as spilling out of the lubricant from the cassette is prevented.

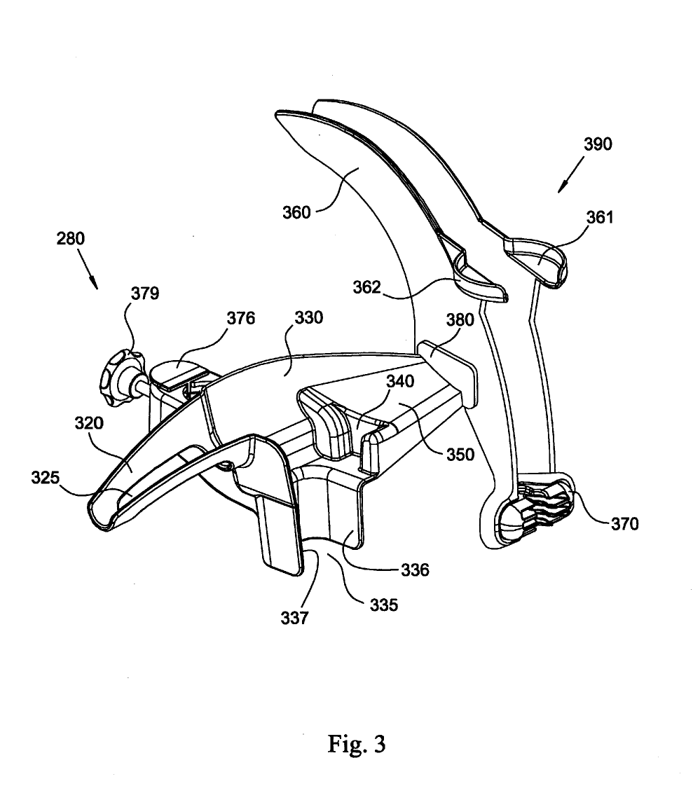

FIGS. 3, 4 and 5 show a first embodiment of the hanger member. In this embodiment a hanger member 280 is provided with a first shoulder portion 320, with an intermediate portion 330, with a carrier portion 350 and with a second shoulder portion 360. The first shoulder portion, the intermediate portion and the carrier portion are formed as an integral part. In practice the hanger member is manufactured by injection molding from a composite plastic material, e.g. Polyamide reinforced by glass fiber.

The first shoulder portion is intended for supporting the umbilical cord and the multichannel. It is configured as an elongate gutter, which can be provided with a window 325. The cross-sectional configuration of the gutter, the gutter width and the gutter length is selected to enable convenient support for the umbilical cord and for the multichannel.

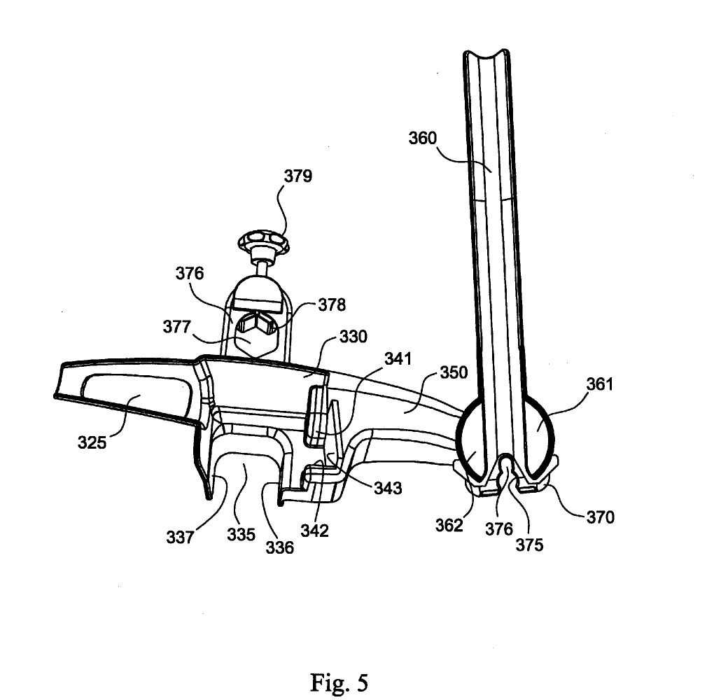

The intermediate portion is intended for receiving the operating handle such that it can be retained in a hanging position. The intermediate portion is configured with a recess, and the lower part of the recess has an opening 335 for passing the lower part of the handle and the insertion tube therethrough. The upper part of the recess has a supporting region 340 for supporting the control buttons of the operating handle. The supporting region is delimited by lateral walls 341, 342, 343 (shown in FIG. 5). When the operating handle is received in the recess the walls provide lateral support for the control buttons and prevent the handle from falling dawn when the handle is inadvertently tilted forward, rearward or sideward. The opposite walls 336, 337 of the opening 335 as well as lateral walls 341, 342, 343 of the supporting region 340 are slanted such that any operating handle, irrespective of its size, can be received and reliably hung on the intermediate portion while still being prevented from falling.

The intermediate portion is fitted with a detent arrangement for securing the hanger member on the post member. The detent arrangement is located at a rear side of the intermediate portion and (as shown in FIG. 5) comprises a bracket 376 having an opening 377 and a V-grooved prism 378, which is displaceable across the opening by a threaded detent knob 379. The opening side, which is opposite to the prism, has a V-shape. By virtue of this provision the prism can lock the post member when the detent knob is screwed and a nut 381 provided within the bracket and thus the hanger member would be clampingly securable on the post member at any location along the post member.

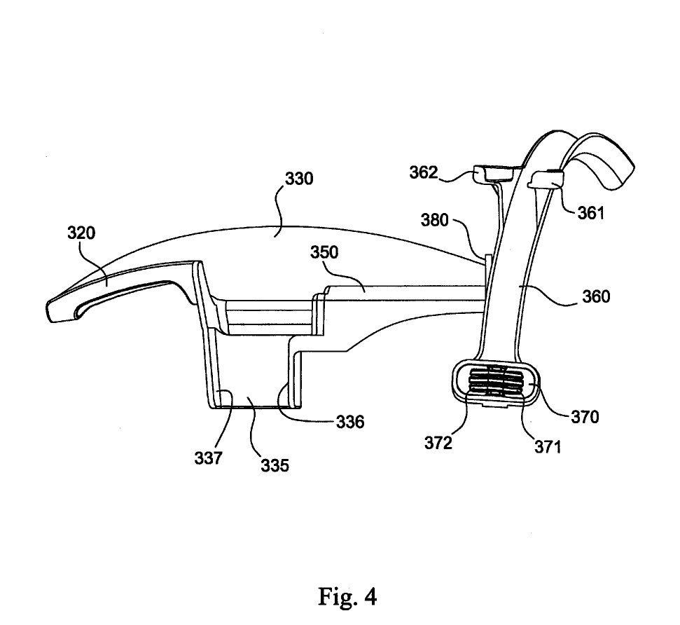

The carrier portion is intended to carry thereon the insertion tube. At the end of the carrier portion there are provided two parallel vertical walls, which define a slot into which the second shoulder portion can be detachably received and secured therein by friction. By virtue of this provision the second shoulder portion is also carried by the carrier portion. One of the vertical walls is seen in FIG. 3 and FIG. 4 and it is designated by reference numeral 380.

The second shoulder portion is intended to retain the distal section of the insertion tube in an erected position, for clamping of the insertion tube and for supporting the cassette in the up-right position. The second shoulder portion is configured as a gutter, which has an arched shape. The radius of curvature of the gutter, its length and its cross-sectional configuration are selected to ensure reliable retention of the insertion tube by the second shoulder portion and stable position of the insertion tube without sliding. The lower end of the second shoulder portion is fitted with a clamp piece 370, which is detachably connectable thereto. The clamp piece is made of a resilient material, e.g. polyurethane and it has a cut-out, which comprises a first rounded opening 375 and a second rounded opening 376. (see FIG. 5) The openings are intended for receiving the insertion tube of the endoscope. The size of the first opening is larger than of the second opening. Both openings are configured and dimensioned to ensure clamping of any insertion tube, when it is entered in the cut-out, irrespective of its outside diameter. Clamping of the insertion tube is achieved due to resiliency of the clamp piece material and due to resiliency achieved by providing the clamp piece with several rows of horizontal fins 371 interspaced by slots 372.

The second shoulder portion is provided with a shelf arrangement 390 (see FIG. 3) for supporting the cassette. The shelf arrangement comprises a couple of arched shelves 361,362, which are formed at opposite sides of the gutter, such that the cassette with the insertion tube extending therealong can be easily placed on the shelves and removed therefrom.

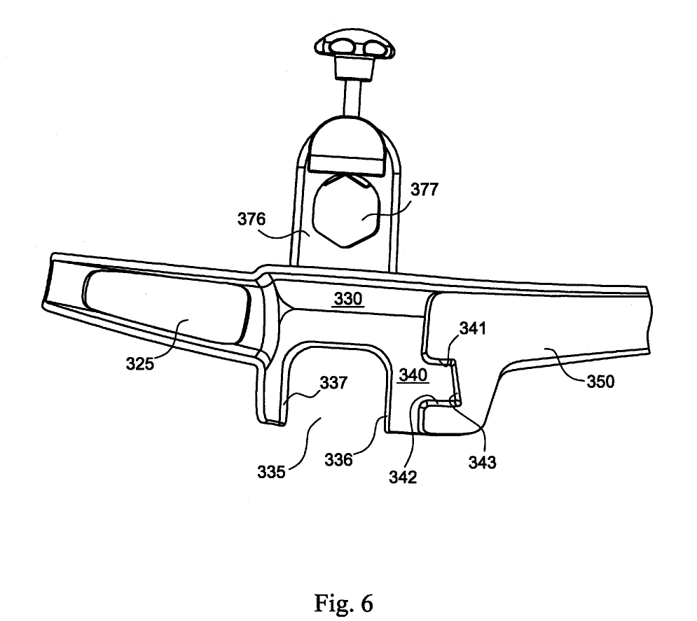

FIG. 6 shows the intermediate portion of the hanger member with recess 335 and support region 340, delimited by lateral walls 341, 342, 343.

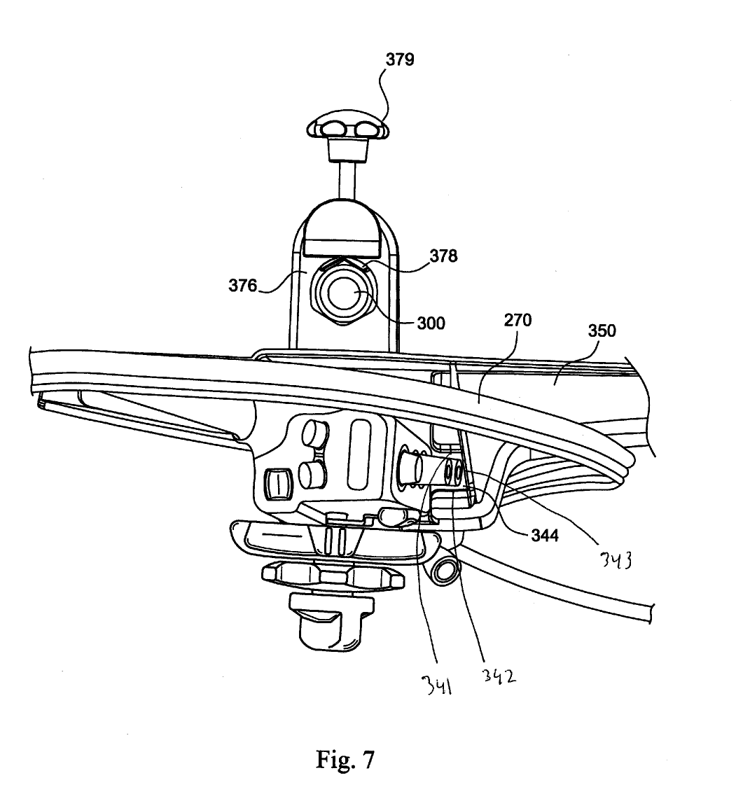

FIG. 7 shows a top view of the operating handle being received in the recess 335 of the intermediate portion 330. It is seen that control knobs 344 of the operating handle 140 are located within support region 340 delimited by lateral walls 341, 342, 343.

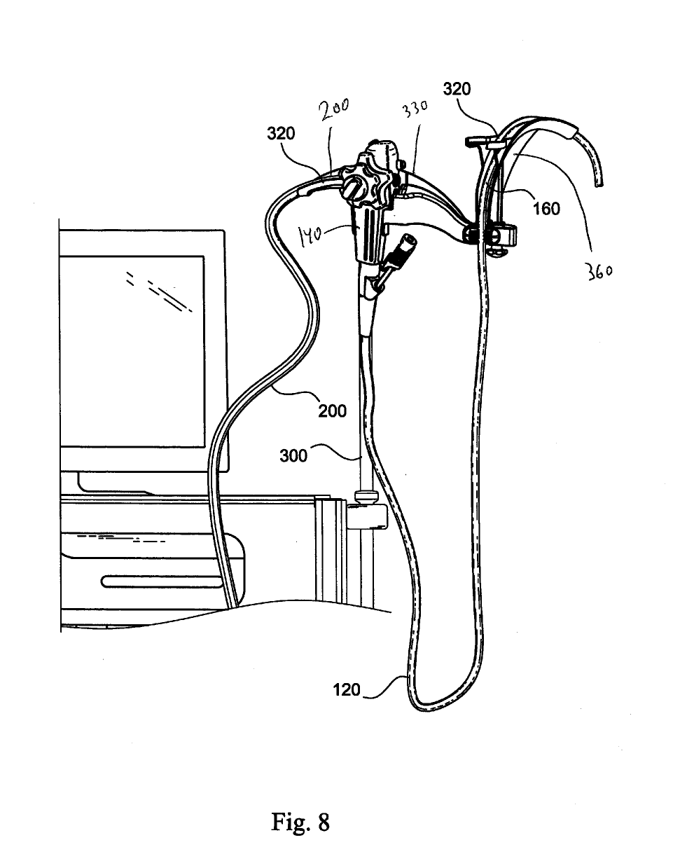

FIG. 8 shows the endoscope resting on the stand of the invention. It is seen that operating handle 140 is supported by the intermediate portion 330, the umbilical cord 200 is resting on the first shoulder portion 320 and the distal section 160 of the insertion tube is supported on the second shoulder portion 360 while the proximal section 120 of the insertion tube is directed down.

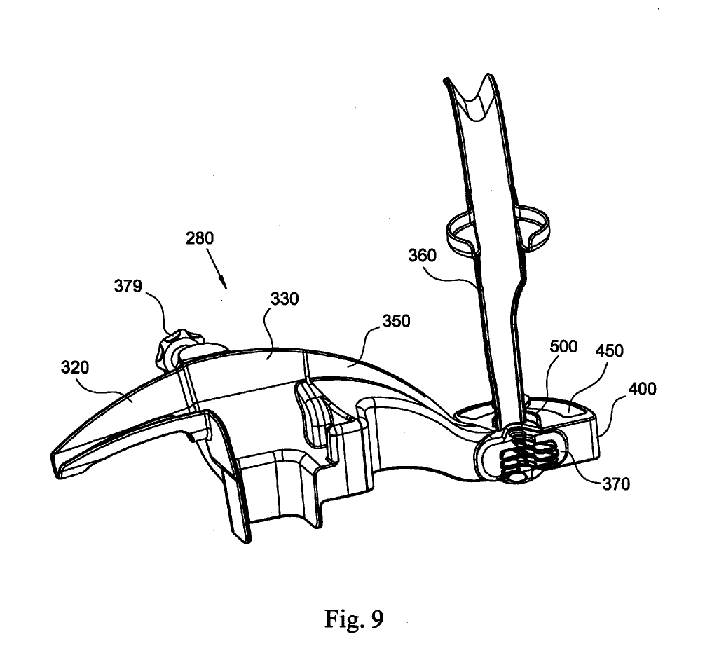

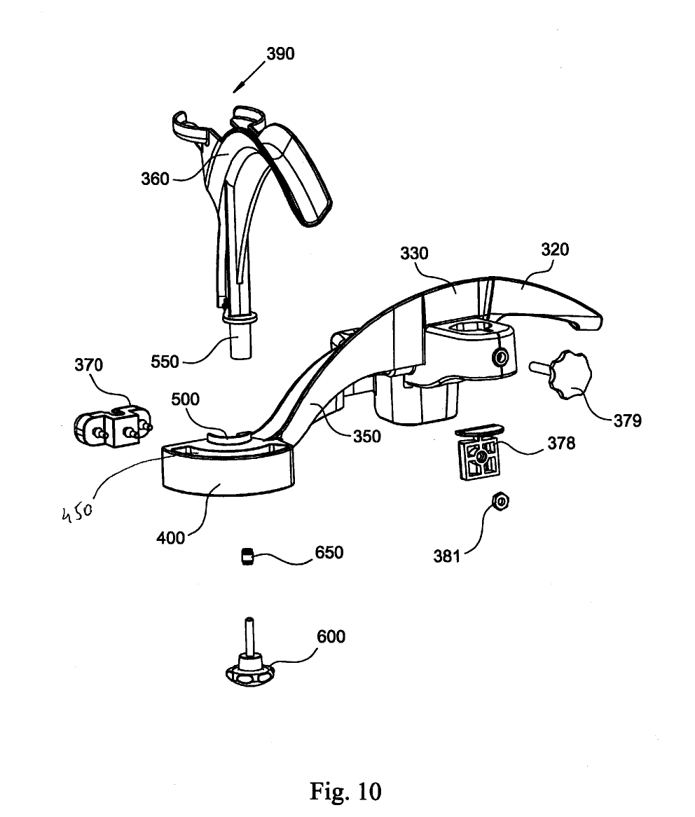

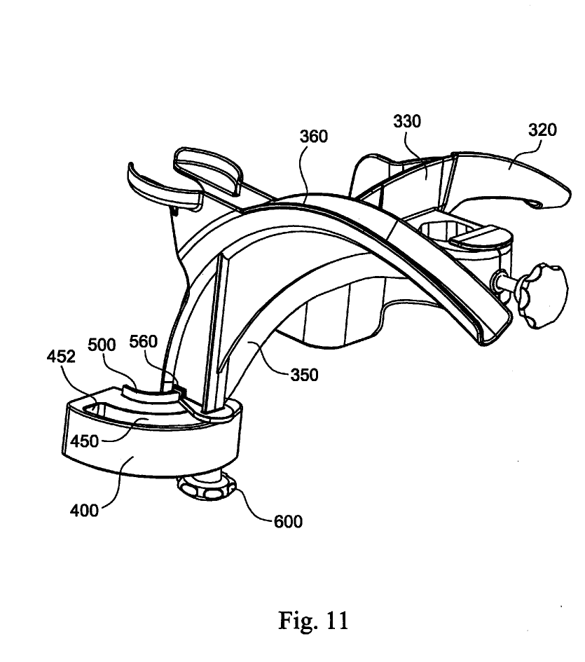

FIGS. 9 and 10 show a second embodiment of the hanger member having mostly the same components as in the first embodiment. The similar components are designated by the same reference numerals as in the first embodiment. In contrast to the first embodiment the carrier portion 350 is provided with an end 400, about which the second shoulder portion 360 can pivot. To make this possible, the end 400 of the carrier portion is provided with an arched slot 450 and a concentric arched guiding wall 500. As seen in FIG. 10 the lower end of the second shoulder portion 360 is fitted with a hollow axle 550, which is dimensioned to enter into the slot 450 and to permit sliding of the axle along the slot such that the second shoulder portion 360 can be pivoted with respect to the carrier portion 350 between closed ends of the slot. By virtue of this provision an operator can adjust the position of the second shoulder portion 360 with respect to the carrier portion 350 such that the placement of the insertion tube on the second shoulder portion would be always convenient and easy, irrespective whether the stand is situated at the right or the left side of the SCU. As seen in FIG. 11 the lower end of the second shoulder portion is provided with a rectangular window 560, which is dimensioned and configured to provide reliable guiding of the arched wall 500 within the window and thus guiding of the second shoulder portion along the slot when the second shoulder portion is pivotally displaced.

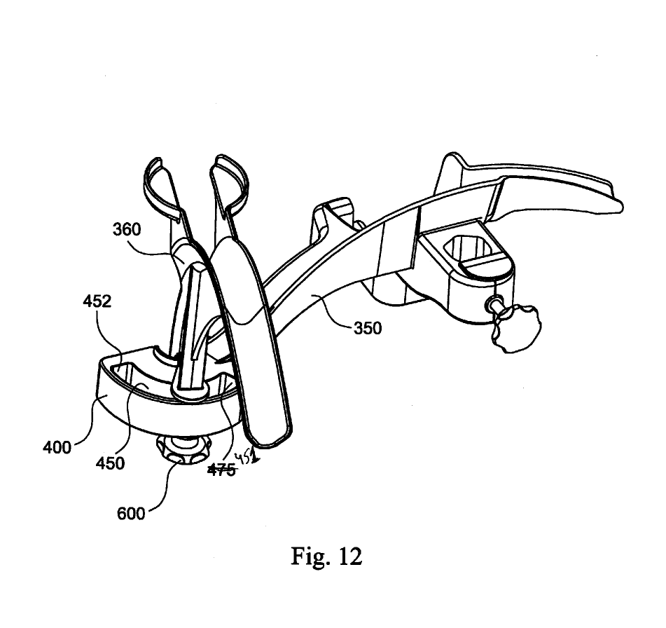

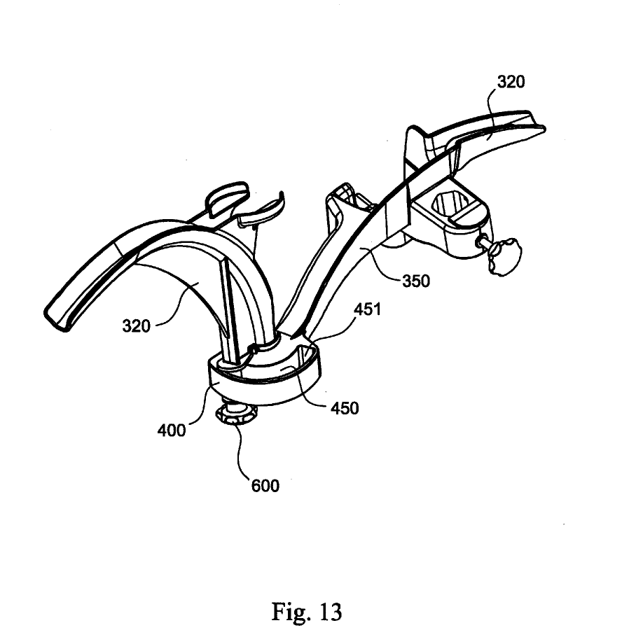

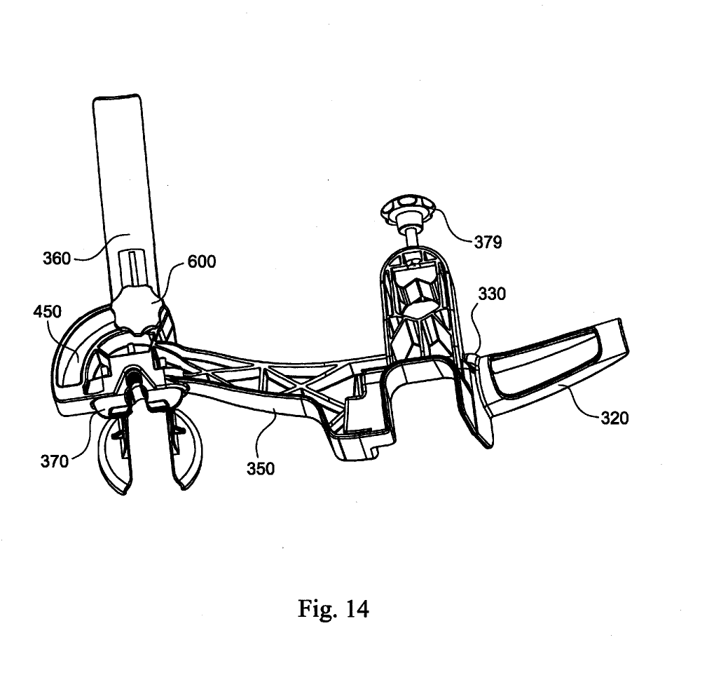

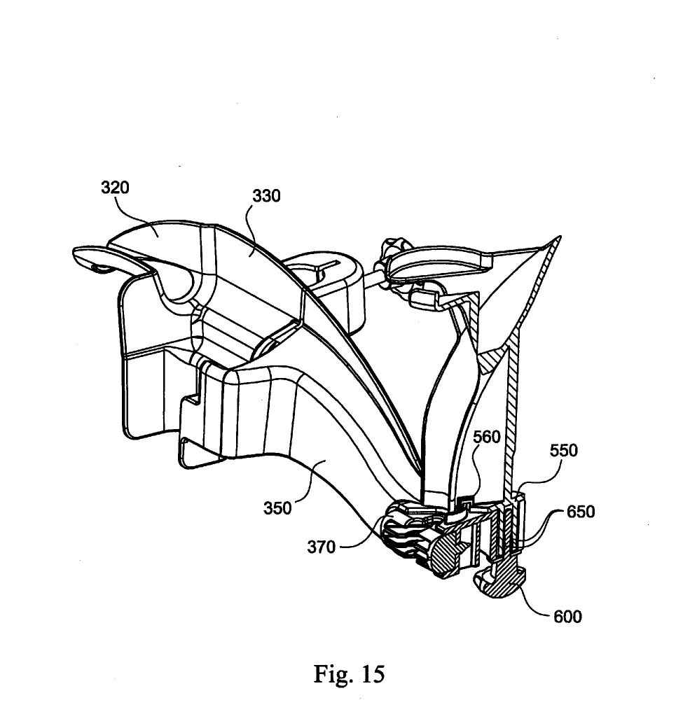

With reference to FIGS. 11-15 it is shown how the second shoulder portion is being pivotally displaced from a first end position near a right closed end 451 of the slot 450 to an intermediate position and then to a second end position near a left closed end 452 of the slot 450. As soon as the operator has pivoted the second shoulder portion adjusted the required position of the second shoulder portion within the slot with respect to the carrier portion, he can secure this position by a threaded detent knob 600, which counteracts with a corresponding threaded bushing 650 (seen in FIG. 15) provided in the hollow axle.

Thus by virtue of the present invention reliable and safe retaining of the operating handle on the hanger member is possible irrespective of the handle size.

Furthermore by virtue of the invention there is provided a possibility for adjusting the height of the hanger member on the post member.

There is provided also a possibility for retaining the insertion tube in a stand-by position when its distal end is in an erect position and the cassette is supported from below. By virtue of this provision spilling out of the lubricant before beginning the endoscopic procedure is prevented.

Furthermore there is provided versatility of clamping since reliable clamping of the insertion tube on the second shoulder portion is possible irrespective of the insertion tube diameter.

Furthermore there is provided convenience in operation since the stand can be disposed at either the left or right side of the system control unit and since the bending section of the insertion tube can be kept in an elevated position, which height can be adjusted according to the operator's height and his both hands can be left free.

It should be appreciated that the present invention is not limited to the above-described embodiments and that changes and one ordinarily skilled in the art can make modifications without deviation from the scope of the invention, as will be defined in the appended claims. So, for example the stand of the invention would be suitable for any endoscopic apparatus provided with flexible insertion tube irrespective whether it is covered or not by a protective sleeve and not only for the apparatus provided with insertion tube coverable by inflatable sleeve deployed within a cassette.

When used in the following claims, the meaning of terms “comprise”, “include”, “have” and their conjugates is “including but not limited to”.

It should also be appreciated that the features disclosed in the foregoing description, and/or in the following claims, and/or in the accompanying drawings may, both separately and in any combination thereof, are material for realizing the present invention in diverse forms thereof.

We claim:

1. A stand for supporting at least an operating handle and an insertion tube of an endoscopic apparatus, said stand comprising a hanger member supported on a post member, said hanger member comprising
a first shoulder portion for supporting an umbilical cord of the endoscopic apparatus,
an intermediate portion for supporting the operating handle,
a carrier portion for supporting the insertion tube, and
a second shoulder portion for retaining the insertion tube in an up-right position.

2. The stand as defined in claim 1, wherein said second shoulder portion being detachably connectable to the carrier portion.

3. The stand as defined in claim 1, wherein the intermediate portion being rigidly connected to the first shoulder portion and to the carrier portion.

4. The stand as defined in claim 1, wherein said second shoulder portion is pivotable with respect to the carrier portion.

5. The stand as defined in claim 1, wherein said first shoulder portion is configured as a gutter.

6. The stand as defined in claim 5, wherein the first shoulder portion is provided with a window made in the bottom of the gutter.

7. The stand as defined in claim 1, wherein the intermediate portion being provided with a recess for receiving the handle, said recess being open from below to allow passing therethrough of the insertion tube and said recess is provided with a support region for receiving control buttons of the operating handle and with walls providing lateral support for the control buttons.

8. The stand as defined in claim 7, wherein the recess, the support region and the walls being configured and dimensioned to allow receiving of an operating handle irrespective of its manufacturing size.

9. The stand as defined in claim 1, wherein the intermediate portion being releasably securable on the post member.

10. The stand as defined in claim 9, wherein the intermediate portion being securable by a threaded detent knob provided at the hanger member.

11. The stand as defined in claim 1, wherein the second shoulder portion being configured as a gutter having arched shape and its radius of curvature and its cross-sectional configuration being selected to provide friction between the gutter and the insertion tube and to allow reliable supporting of the insertion tube on the second shoulder portion without sliding.

12. The stand as defined in claim 11, wherein the second shoulder portion being provided with a clamp piece which is detachably affixed thereto.

13. The stand as defined in claim 12, wherein the clamp piece being made of a resilient material and being provided with a cut-out, the dimension and configuration of said cut-out being selected to allow inserting of the insertion tube thereinto and its clamping due to the resiliency of the clamp piece.

14. The stand as defined in claim 4, wherein the second shoulder portion is pivotally displaceable with respect to the carrier portion.

15. The stand as defined in claim 14, wherein the second shoulder portion being securable in a position on the carrier portion by a detent knob.

16. The stand as defined in claim 1, wherein said endoscopic apparatus is a colonoscopic apparatus.

17. The stand as defined in claim 16, wherein said colonsocopic apparatus being provided with a cassette loaded with a lubricant for lubricating the insertion tube and said second shoulder portion being provided with a shelf arrangement for placing the cassette thereon and for supporting the cassette in an upright position.

18. The stand as defined in claim 13, wherein the clamp piece being made of polyurethane.

19. The stand as defined in claim 18, wherein the cut-out is provided with a first rounded opening and with a second rounded opening, said openings being configured and dimensioned to ensure clamping of any insertion tube, when it is entered in the cut-out, irrespective of its outside diameter.

20. The stand as defined in claim 19, wherein the clamp piece being provided with several rows of fins interspaced by slots.
